SEQUENCE LISTING (46 sequences)

SEQ ID NO: 1 - protein, 5 aa, Homo sapiens
  SOURCE 1..5: /mol_type=“protein” /organism=“Homo sapiens”
NYWMH

SEQ ID NO: 2 - protein, 17 aa, Homo sapiens
  SOURCE 1..17: /mol_type=“protein” /organism=“Homo sapiens”
GISGWSSWTYYADSVKG

SEQ ID NO: 3 - protein, 13 aa, Homo sapiens
  SOURCE 1..13: /mol_type=“protein” /organism=“Homo sapiens”
FGIDAYTKVYFDY

SEQ ID NO: 4 - protein, 11 aa, Homo sapiens
  SOURCE 1..11: /mol_type=“protein” /organism=“Homo sapiens”
SGDNIPLKYVS

SEQ ID NO: 5 - protein, 7 aa, Homo sapiens
  SOURCE 1..7: /mol_type=“protein” /organism=“Homo sapiens”
DDNKRPS

SEQ ID NO: 6 - protein, 9 aa, Homo sapiens
  SOURCE 1..9: /mol_type=“protein” /organism=“Homo sapiens”
QSWASTGVV

SEQ ID NO: 7 - protein, 122 aa, Homo sapiens
  SOURCE 1..122: /mol_type=“protein” /organism=“Homo sapiens”
QVELVESGGGLVQPGGSLRLSCAASGFTFSNYWMHWVRQAPGKGLEWVSGISGWSSWTYYADSVKGRFTISRDNSKNTLYLQMNSLRAEDTAVYYCARFGIDAYTKVYFDYWGQGTLVTVSS

SEQ ID NO: 8 - protein, 107 aa, Homo sapiens
  SOURCE 1..107: /mol_type=“protein” /organism=“Homo sapiens”
DIELTQPPSVSVAPGQTARISCSGDNIPLKYVSWYQQKPGQAPVLVIHDDNKRPSGIPERFSGSNSGNTATLTISGTQAEDEADYYCQSWASTGVVFGGGTKLTVLG

SEQ ID NO: 9 - protein, 452 aa, Homo sapiens
  SOURCE 1..452: /mol_type=“protein” /organism=“Homo sapiens”
QVELVESGGGLVQPGGSLRLSCAASGFTFSNYWMHWVRQAPGKGLEWVSGISGWSSWTYYADSVKGRFTISRDNSKNTLYLQMNSLRAEDTAVYYCARFGIDAYTKVYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 212 aa, Homo sapiens
  SOURCE 1..212: /mol_type=“protein” /organism=“Homo sapiens”
DIELTQPPSVSVAPGQTARISCSGDNIPLKYVSWYQQKPGQAPVLVIHDDNKRPSGIPERFSGSNSGNTATLTISGTQAEDEADYYCQSWASTGVVFGGGTKLTVLGQPKAAPSVTLFPPSSEELQANKATLVCLISDFYPGAVTVAWKGDSSPVKAGVETTTPSKQSNNKYAASSYLSLTPEQWKSHRSYSCQVTHEGSTVEKTVAPTECS

SEQ ID NO: 11 - protein, 5 aa, Homo sapiens
  SOURCE 1..5: /mol_type=“protein” /organism=“Homo sapiens”
NYWMH

SEQ ID NO: 12 - protein, 17 aa, Homo sapiens
  SOURCE 1..17: /mol_type=“protein” /organism=“Homo sapiens”
GISGWSSWTYYADSVKG

SEQ ID NO: 13 - protein, 13 aa, Homo sapiens
  SOURCE 1..13: /mol_type=“protein” /organism=“Homo sapiens”
FGIDAYTKVYFDY

SEQ ID NO: 14 - protein, 11 aa, Homo sapiens
  SOURCE 1..11: /mol_type=“protein” /organism=“Homo sapiens”
SGDNIPLKYVS

SEQ ID NO: 15 - protein, 7 aa, Homo sapiens
  SOURCE 1..7: /mol_type=“protein” /organism=“Homo sapiens”
DDNKRPS

SEQ ID NO: 16 - protein, 11 aa, Homo sapiens
  SOURCE 1..11: /mol_type=“protein” /organism=“Homo sapiens”
SSWDTLDIFNV

SEQ ID NO: 17 - protein, 122 aa, Homo sapiens; identical to SEQ ID NO: 7
  SOURCE 1..122: /mol_type=“protein” /organism=“Homo sapiens”

SEQ ID NO: 18 - protein, 109 aa, Homo sapiens
  SOURCE 1..109: /mol_type=“protein” /organism=“Homo sapiens”
DIELTQPPSVSVAPGQTARISCSGDNIPLKYVSWYQQKPGQAPVLVIHDDNKRPSGIPERFSGSNSGNTATLTISGTQAEDEADYYCSSWDTLDIFNVFGGGTKLTVLG

SEQ ID NO: 19 - protein, 452 aa, Homo sapiens; identical to SEQ ID NO: 9
  SOURCE 1..452: /mol_type=“protein” /organism=“Homo sapiens”

SEQ ID NO: 20 - protein, 214 aa, Homo sapiens
  SOURCE 1..214: /mol_type=“protein” /organism=“Homo sapiens”
DIELTQPPSVSVAPGQTARISCSGDNIPLKYVSWYQQKPGQAPVLVIHDDNKRPSGIPERFSGSNSGNTATLTISGTQAEDEADYYCSSWDTLDIFNVFGGGTKLTVLGQPKAAPSVTLFPPSSEELQANKATLVCLISDFYPGAVTVAWKGDSSPVKAGVETTTPSKQSNNKYAASSYLSLTPEQWKSHRSYSCQVTHEGSTVEKTVAPTECS

SEQ ID NO: 21 - protein, 5 aa, Homo sapiens
  SOURCE 1..5: /mol_type=“protein” /organism=“Homo sapiens”
NYWMH

SEQ ID NO: 22 - protein, 17 aa, Homo sapiens
  SOURCE 1..17: /mol_type=“protein” /organism=“Homo sapiens”
GISGWSSWTYYADSVKG

SEQ ID NO: 23 - protein, 13 aa, Homo sapiens
  SOURCE 1..13: /mol_type=“protein” /organism=“Homo sapiens”
FGIDAYTKVYFDY

SEQ ID NO: 24 - protein, 11 aa, Homo sapiens
  SOURCE 1..11: /mol_type=“protein” /organism=“Homo sapiens”
SGDNIPLKYVS

SEQ ID NO: 25 - protein, 7 aa, Homo sapiens
  SOURCE 1..7: /mol_type=“protein” /organism=“Homo sapiens”
DDNKRPS

SEQ ID NO: 26 - protein, 11 aa, Homo sapiens
  SOURCE 1..11: /mol_type=“protein” /organism=“Homo sapiens”
QSYDYFPKFVV

SEQ ID NO: 27 - protein, 122 aa, Homo sapiens; identical to SEQ ID NO: 7
  SOURCE 1..122: /mol_type=“protein” /organism=“Homo sapiens”

SEQ ID NO: 28 - protein, 109 aa, Homo sapiens
  SOURCE 1..109: /mol_type=“protein” /organism=“Homo sapiens”
DIELTQPPSVSVAPGQTARISCSGDNIPLKYVSWYQQKPGQAPVLVIHDDNKRPSGIPERFSGSNSGNTATLTISGTQAEDEADYYCQSYDYFPKFVVFGGGTKLTVLG

SEQ ID NO: 29 - protein, 452 aa, Homo sapiens; identical to SEQ ID NO: 9
  SOURCE 1..452: /mol_type=“protein” /organism=“Homo sapiens”

SEQ ID NO: 30 - protein, 214 aa, Homo sapiens
  SOURCE 1..214: /mol_type=“protein” /organism=“Homo sapiens”
DIELTQPPSVSVAPGQTARISCSGDNIPLKYVSWYQQKPGQAPVLVIHDDNKRPSGIPERFSGSNSGNTATLTISGTQAEDEADYYCQSYDYFPKFVVFGGGTKLTVLGQPKAAPSVTLFPPSSEELQANKATLVCLISDFYPGAVTVAWKGDSSPVKAGVETTTPSKQSNNKYAASSYLSLTPEQWKSHRSYSCQVTHEGSTVEKTVAPTECS

SEQ ID NO: 31 - protein, 5 aa, Homo sapiens
  SOURCE 1..5: /mol_type=“protein” /organism=“Homo sapiens”
SYWMS

SEQ ID NO: 32 - protein, 17 aa, Homo sapiens
  SOURCE 1..17: /mol_type=“protein” /organism=“Homo sapiens”
SITSYGSFTYYADSVKG

SEQ ID NO: 33 - protein, 8 aa, Homo sapiens
  SOURCE 1..8: /mol_type=“protein” /organism=“Homo sapiens”
NMYTHFDS

SEQ ID NO: 34 - protein, 13 aa, Homo sapiens
  SOURCE 1..13: /mol_type=“protein” /organism=“Homo sapiens”
SGSSSNIGSNSVS

SEQ ID NO: 35 - protein, 7 aa, Homo sapiens
  SOURCE 1..7: /mol_type=“protein” /organism=“Homo sapiens”
DNSKRPS

SEQ ID NO: 36 - protein, 11 aa, Homo sapiens
  SOURCE 1..11: /mol_type=“protein” /organism=“Homo sapiens”
QSRDTYGYYWV

SEQ ID NO: 37 - protein, 117 aa, Homo sapiens
  SOURCE 1..117: /mol_type=“protein” /organism=“Homo sapiens”
QVELVESGGGLVQPGGSLRLSCAASGFTFTSYWMSWVRQAPGKGLELVSSITSYGSFTYYADSVKGRFTISRDNSKNTLYLQMNSLRAEDTAVYYCARNMYTHFDSWGQGTLVTVSS

SEQ ID NO: 38 - protein, 111 aa, Homo sapiens
  SOURCE 1..111: /mol_type=“protein” /organism=“Homo sapiens”
DIVLTQPPSVSGAPGQRVTISCSGSSSNIGSNSVSWYQQLPGTAPKLLIYDNSKRPSGVPDRFSGSKSGTSASLAITGLQSEDEADYYCQSRDTYGYYWVFGGGTKLTVLG

SEQ ID NO: 39 - protein, 447 aa, Homo sapiens
  SOURCE 1..447: /mol_type=“protein” /organism=“Homo sapiens”
QVELVESGGGLVQPGGSLRLSCAASGFTFTSYWMSWVRQAPGKGLELVSSITSYGSFTYYADSVKGRFTISRDNSKNTLYLQMNSLRAEDTAVYYCARNMYTHFDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 40 - protein, 216 aa, Homo sapiens
  SOURCE 1..216: /mol_type=“protein” /organism=“Homo sapiens”
DIVLTQPPSVSGAPGQRVTISCSGSSSNIGSNSVSWYQQLPGTAPKLLIYDNSKRPSGVPDRFSGSKSGTSASLAITGLQSEDEADYYCQSRDTYGYYWVFGGGTKLTVLGQPKAAPSVTLFPPSSEELQANKATLVCLISDFYPGAVTVAWKGDSSPVKAGVETTTPSKQSNNKYAASSYLSLTPEQWKSHRSYSCQVTHEGSTVEKTVAPTECS

SEQ ID NO: 41 - protein, 121 aa, Homo sapiens
  SOURCE 1..121: /mol_type=“protein” /organism=“Homo sapiens”
QVQLVQSGAEVKKPGASVKVSCKASGYTFTSYDINWVRQATGQGLEWMGWMNPNSGNTGYAQKFQGRVTMTRDTSISTAYMELSSLRSEDTAVYYCARDPYYYYYGMDVWGQGTTVTVSSA

SEQ ID NO: 42 - protein, 112 aa, Homo sapiens
  SOURCE 1..112: /mol_type=“protein” /organism=“Homo sapiens”
QSVLTQPPSVSAAPGQKVTISCSGSSSNIENNHVSWYQQLPGTAPKLLIYDNNKRPSGIPDRFSGSKSGTSATLGITGLQTGDEADYYCETWDTSLSAGRVFGGGTKLTVLG

SEQ ID NO: 43 - protein, 121 aa, Homo sapiens
  SOURCE 1..121: /mol_type=“protein” /organism=“Homo sapiens”
QVQLVQSGAEVKKPGASVKVSCKASGYTFTSYDINWVRQATGQGLEWMGWMNPNSGNTGYAQKFQGRVTMTRNTSISTAYMELSSLRSEDTAVYYCARDPYYYYYGMDVWGQGTTVTVSSA

SEQ ID NO: 44 - protein, 112 aa, Homo sapiens; identical to SEQ ID NO: 42
  SOURCE 1..112: /mol_type=“protein” /organism=“Homo sapiens”

SEQ ID NO: 45 - protein, 450 aa, Homo sapiens
  SOURCE 1..450: /mol_type=“protein” /organism=“Homo sapiens”
EVQLLESGGGLVQPGGSLRLSCTASGFTFSSYAMNWVRQAPGKGLEWVSAISGSGGTTFYADSVKGRFTISRDNSRTTLYLQMNSLRAEDTAVYYCAKDLGWSDSYYYYYGMDVWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPG

SEQ ID NO: 46 - protein, 214 aa, Homo sapiens
  SOURCE 1..214: /mol_type=“protein” /organism=“Homo sapiens”
DIQMTQFPSSLSASVGDRVTITCRASQGIRNDLGWYQQKPGKAPKRLIYAASRLHRGVPSRFSGSGSGTEFTLTISSLQPEDFATYYCLQHNSYPCSFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2015/0209426 A1
(43) Pub. Date: Jul. 30, 2015

(54) TREATMENT OF CANCER

(71) Applicant: Boehringer Ingelheim International GmbH, Ingelheim am Rhein (DE)

(72) Inventors: Thomas BOGENRIEDER, Landshut (DE); Ulrike WEYER-CZERNILOFSKY, Baden (AT)

(21) Appl. No.: 14/603,503

(22) Filed: Jan. 23, 2015

(30) Foreign Application Priority Data
Jan. 24, 2014 (EP) 14152416.5

(51) Int. Cl. A61K 39/395; A61K 31/439; A61K 31/566

(52) U.S. Cl. CPC A61K 39/3955; A61K 31/566; A61K 31/439; A61K 2039/505

(57) ABSTRACT

A method for treating breast cancer with an insulin-like growth factor (IGF) receptor antagonist in combination with exemestane and everolimus.

The present invention relates to the pharmaceutical treatment of patients with breast cancer.

### BACKGROUND OF THE INVENTION

Breast cancer is the most common malignancy in women worldwide. It is estimated that more than 1.6 million new cases of breast cancer occurred globally among women in 2010 (Forouzanfar M, Foreman K, Delossantos A M, Lozano R, Lopez A D, Murray C J L, et al. Lancet 378, 1461-1484 (2011). Even though death rates have fallen steadily since 1990, reflecting improvements in early detection and treatment, currently breast cancer is the second leading cause of cancer related death in women. This high death rate reflects the limited effectiveness of current therapeutic options, particularly in patients with advanced disease.

Approximately 75% of primary breast cancers are positive for hormone receptor (HR+). These cancers express Estrogen Receptor (ER) and/or Progestone Receptor (PgR). Therapies directed at endocrine receptors are important treatment option. For postmenopausal HR+ breast cancer, an aromatase inhibitor (AI), such as letrozole and anastrozole, is the recommended first-line therapy for management. Unfortunately, not all patients have a response to first-line endocrine therapy (primary or de novo resistance), and even patients who have a response will eventually relapse (acquired resistance). On disease progression, second-line treatment options include other classes of aromatase inhibitors (steroidal or nonsteroidal) and the estrogen-receptor (ER) antagonists fulvestrant and tamoxifen (Villarreal-Garza C, Cortes J, Andre F, Verma S. Ann Oncol 23 (10), 2526-2535 (2012).

De novo and acquired resistance to endocrine therapy presents a major challenge in the management of HR+ breast cancer. The mammalian target of rapamycin (mTOR) pathway has been shown to play an important role in the resistance to endocrine therapy. Two recently published reports showed that a novel mTOR inhibitor everolimus combined with endocrine therapies were of benefit. In hormone refractory, hormone receptor-positive, HER2-negative metastatic breast cancer patients, tamoxifen plus everolimus resulted in increased clinical benefit compared to tamoxifen alone with improved time to progression (PFS 8.6 vs. 4.5 months) and overall survival (55% reduction in the risk of death associated with combination therapy, HR, 0.45; 95% CI, 0.24 to 0.81; exploratory P=007) (Bachelot T, Bourgier C, Cropet C, Ray-Coquard I, Ferrero J M, Freyer G, et al. J Clin Oncol 30 (22), 2718-2724 (2012). In a similar patient population, the Phase III Breast Cancer Trial of Oral Everolimus 2 (BOLERO-2) (Baselga J, Campone M, Piccart M, Burris H A, Rugo H S, Sahmoud T et al. N Engl J Med 2012; 366(6): 520-529) demonstrated that treatment with everolimus plus exemestane more than doubled median progression-free survival to 7.8 months compared with 3.2 months for those treated with exemestane alone (hazard ratio, 0.45; 95% confidence interval, 0.38-0.54; one-sided log rank P<0.0001) by local investigator assessment. The overall response rate was also improved compared to exemestane alone (12.6% vs. 1.7%). Based on the result of this trial, everolimus, as the first drug in the mTOR class, was approved by the regulatory agency for the treatment of postmenopausal women with advanced hormone receptor-positive, HER2− negative breast cancer (advanced HR+BC) in combination with exemstane, after failure of treatment with letrozole or anastrozole.

Insulin-like growth factor-1 (IGF-1; a 70 amino-acid polypeptide) and insulin-like growth factor-2 (IGF-2; a 67 amino-acid polypeptide) are 7.5-kD soluble factors present in serum that can potently stimulate the growth of many mammalian cells (reviewed by Pollack et al., Nature Rev. Can. 4: 505-518, 2004). On secretion into the bloodstream the IGFs form complexes with the IGFBPs which protect them from proteolytic degradation in the serum en route to their target tissues and prevents their association with the IGF receptors. IGFs are also known to be secreted in an autocrine or paracrine manner in target tissues themselves. This is known to occur during normal fetal development where the IGFs play a key role in the growth of tissues, bone and organs. It is also seen in many cancer tissues where there is thought to be paracrine signaling between tumour cells and stromal cells or autocrine IGF production by the tumour cells themselves (reviewed by LeRoith D, Experimental Diab. Res. 4: 205-212, 2003).

IGF-1 and IGF-2 are able to bind to the IGF-1 receptor (IGF-1R) expressed on many normal tissues, which functionally is a 460 kD heterotetramer consisting of a dimerised alpha- and beta-subunit, with similar affinities (Rubin et al., Lab. Invest. 73: 311-31, 1995). IGF-2 can also bind to the IGF-2 receptor, which is thought to prevent IGF-2 from binding and signaling through the IGF-1R. In this respect the IGF-2R has been demonstrated to be a tumour suppressor protein. The IGF-1R is structurally similar to the insulin receptor which exists in two forms, IR-A and IR-B, which differ by an alternatively spliced 12 amino acid exon deletion in the extracellular domain of IR-A. IR-B is the predominant IR isoform expressed in most normal adult tissues where it acts to mediate the effects of insulin on metabolism. IR-A on the other hand is known to be highly expressed in developing fetal tissues but not in adult normal tissues. Recent studies have also shown that IR-A, but not IR-B, is highly expressed in some cancers. The exon deletion in IR-A has no impact on insulin binding but does cause a small conformational change that allows IGF-2 to bind with much higher affinity than for IR-B (Frasca et al., Mol. Cell. Biol. 19: 3278-88, 1999; Pandini et al., J. Biol. Chem. 277: 39684-95, 2002). Thus, because of it's expression in cancer tissues and increased propensity for IGF-2 binding, IR-A may be as important as IGF1-R in mediating the mitogenic effects of IGF-2 in cancer.

Binding of the IGFs to IGF-1R triggers a complex intracellular signaling cascade which results in activation of proteins that stimulate proliferation and survival (reviewed by Pollack et al., Nature Rev. Can. 4: 505-518, 2004).

There is a very large body of scientific, epidemiological and clinical literature implicating a role for the IGFs in the development, progression and metastasis of many different cancer types (reviewed by Jerome et al., End. Rel. Cancer 10: 561-578, 2003; and Pollack et al., Nature Rev. Can. 4: 505-518, 2004).

Preclinical and clinical data indicated that aberrant regulation of the IGF system is attributed to the pathogenesis of breast cancer and also contributes to various stages of breast carcinogenesis. IGF-1R over-expression is common in breast cancer cell lines and fresh tumor biopsies (Cullen K J, Yee D, Sly W S, Perdue J, Hampton B, Lippman M E, Rosen N Cancer Res 50 (1), 48-53 (1990), Yang Y, Yee D. J Mammary Gland Biol Neoplasia 17 (3/4), 251-261 (2012), Peyrat J P, Bonneterre J, Beuscart R, Dijane J, Demaille A. Cancer Res 48 (22), 6429-6433 (1988), and IGF activity captured in a microarray signature has been associated with poor clinical outcome (Zardavas, D, Basalga J and Piccart M. Nat Rev Clin Oncol. 2013 April; 10(4):191-210.). Bidirectional crosstalk between the oestrogen and the IGF signalling pathways is well documented (Clarke R B, Howell A, Anderson E. Br J Cancer 75 (2), 251-257 (1997), Hamelers I H L, Schaik RFMA van, Teeffelen HAAM van, Sussenbach J S, Steenbergh P H. Exp Cell Res 273, 107-117) with activation of the latter mediating endocrine resistance (Wiseman L R, Johnson M D, Wakeling A E, Lykkesfeldt A E, May F E B, Westley B R. Eur J Cancer (A) 29 (16), 2256-2264 (1993). In ER+ breast cancer resistant to hormonal therapy, InsR-A isoform is the predominant insulin receptor, suggesting an important role for IGF-2 signaling (Bachelot T, Bourgier C, Cropet C, Ray-Coquard I, Ferrero J M, Freyer G, et al. J Clin Oncol 30 (22), 2718-2724 (2012). Therefore, the IGF signalling network represents a promising target in advanced breast cancer.

Currently there are three different strategies inhibiting IGF pathways including anti-receptor monoclonal antibodies (ganitumab, cixutumumab, and dalotuzumab), tyrosine kinase inhibitors (TKI, including dual IGF-1R and InsR tyrosine kinase inhibitors BMS-754807, KW2450, and linsitinib), and anti-IGF ligand antibodies (dusigitumab (MEDI-573, Astra Zeneca/MedImmune). These agents are now being tested in the clinic either as monotherapy or in combination with cytotoxic agents and/or other molecularly targeted agents.

The major advantage of neutralizing antibodies for both IGF-1 and IGF-2 is that the sequestration of the ligands ensures that receptor activation by IGF-1 or IGF-2 does not occur, and eliminates the possibility of activation of the InsR-A by IGF-2. Hence it offers a balanced approach with therapeutic potential in a variety of cancers, with few of the pitfalls of targeting the IGF-1R with monoclonal antibodies (mAbs).

A potential mechanism of resistance to mTOR inhibitor therapy is the induction of AKT phosphorylation, which is frequently observed in both preclinical and clinical studies (Sun S Y, Rosenberg L M, Wang X, Zhou Z, Yue P, Fu H, et al. Cancer Res. 2005 Aug. 15; 65(16):7052-8; Tabernero J, Rojo F, Calvo E, Burris H, Judson I, Hazell K, et al. J Clin Oncol 2008; 26(10):1603-1610; Wan X, Harkavy B, Shen N, Grohar P, Heiman L J. Oncogene 26, 1932-1940 (2007); O'Reilly K E, Rojo F, She Q B, Solit D, Mills G B, Smith D, et al. Cancer Res 66 (3), 1500-1508 (2006)). Furthermore, up-regulation of AKT activity is dependent on Insulin-like growth factor (IGF)/Insulin like growth factor type 1 receptor (IGF-1R) signalling. It has been shown in tumors that inhibition of mTOR with rapalogs releases a negative feedback loop on growth factor receptors, including the insulin-like growth factor-1 receptor/insulin receptor substrate (IRS)-1 complex, resulting in activation of IGF-1R signaling and ultimately phosphorylation of AKT, which in turn could potentially counteract the anti-tumor effects of mTOR inhibitors. The activation may be prevented if IGF signaling is blocked simultaneously (Higgins M J, Baselga J. J Clin Invest 121 (10), 3797-3803 (2011).

Against this background, the present inventors decided to combine a human anti-IGF antibody with exemestane and everolimus. They have surprisingly found that a triple combination of the human anti-IGF antibody with exemestane and everolimus has a clear advantage in affecting the growth of a breast cancer cell line than the double combination of exemestane and everolimus. Until the present invention it had not been disclosed or contemplated to combine human anti-IGF antibody with exemestane and everolimus to treat patients with breast cancer.

The present application relates to an advantageous combination of a human anti-IGF antibody with exemestane and everolimus in patients with breast cancer.

### BRIEF DESCRIPTION OF THE FIGURES

FIG. 1. Effect of a triple combination of IGF Ab 60833, everolimus and exemestane, against a double combination of everolimus and exemestane. The figure shows a comparison of the double combination of everolimus and exemestane (top panel) against the triple combination of IGF Ab 60833, everolimus and exemestane (bottom panel). FIG. 1 A.: Double combination−Everolimus+Exemestane “Campaign “3× combi separated by mAB concentration”, N=3 avg(Bliss CGIₘ) of AlamarBlue_cell line (144h,cell{29164/5000/}) FIG. 1 B.: Triple combination: Everolimus+Exemestane, +60833 (10 nM)“Campaign “3× combi separated by mAB concentration”, N=3 avg(Bliss CGIₘ) of AlamarBlue_cell line (144h,cell{29164/5000/})

FIG. 2. Effect of everolimus and exemestane, alone or in combination, on the in vitro growth of MCF7aro cells. In three independent experiments (A, B, C) MCF7aro cells were treated with different concentrations of everolimus and exemestane, as single agents and in combination. After incubation for 6 days the inhibitory effect on MCF7aro cell growth was quantified using the AlamarBlue® cell viability assay. The data shown in (D) represent the mean values of the three independent experiments.

FIG. 3. Effect of everolimus and exemestane, alone or in combination, combined with 10 nM of IGF Ab 60833, on the in vitro growth of MCF7aro cells. In three independent experiments (A, B, C) MCF7aro cells were treated with different concentrations of everolimus and exemestane, as single agents and in combination, combined with 10 nM of IGF Ab 60833. After incubation for 6 days the inhibitory effect on MCF7aro cell growth was quantified using the AlamarBlue® cell viability assay. The data shown in (D) represent the mean values of the three independent experiments

FIG. 4. Effect of IGF Ab 60833 and everolimus, alone or in combination, on the AKT and S6 phosphorylation status in MCF7aro cells in vitro. MCF7aro cells were treated with 100 nM IGF Ab 60833 and 0.32 nM everolimus as single agents or in combination for 24 hours. Cell lysates prepared from vehicle (DMSO)-treated and inhibitor-treated cells were analysed by Western blotting

FIG. 5. Effect of IGF Ab 60833, exemestane and everolimus, alone or in combination, on induction of apoptosis in MCF7aro cells in vitro. MCF7aro cells were treated with 1 μM IGF Ab 60833, 1 μM exemestane and 1 μM everolimus as single agents or in combination for 72 hours. Cell lysates prepared from vehicle (DMSO)-treated and inhibitor-treated cells were analysed by Western blotting and MSD Apoptosis Panel for induction of PARP cleavage

### BRIEF DESCRIPTION OF THE INVENTION

In one aspect, the present invention pertains to an insulin-like growth factor (IGF) receptor antagonist for use in the treatment of patients with breast cancer in combination with exemestane and everolimus.

In another aspect, the invention relates to a method of treatment of breast cancer comprising administering a therapeutically effective amount of an IGF receptor antagonist to a patient in need thereof, and additionally administering a therapeutically effective amount of an exemestane and everolimus to the same patient.

Preferably the breast cancer is locally advanced or metastatic breast cancer.

### DETAILED DESCRIPTION OF THE INVENTION

The present invention relates to the combination of an insulin-like growth factor (IGF) receptor antagonist with exemestane (Aromasin®) and the rapalog (first generation mTOR inhibitor) everolimus (Afinitor®) in breast cancer, specifically advanced estrogen receptor positive breast cancer. Mammalian target of rapamycin (mTOR) inhibitors mediate AKT activation through a type 1 insulin-like growth factor receptor (IGF-1R)-dependent mechanism. The combination with insulin-like growth factor (IGF) receptor antagonist is thought to enhance mTOR-targeted anticancer activity by modulating resistance to mTOR inhibition by targeting feedback loops.

In one aspect, the present invention pertains to an insulin-like growth factor (IGF) receptor antagonist for use in the treatment of patients with breast cancer in combination with exemestane and everolimus.

Preferably the breast cancer is locally advanced.

Preferably the breast cancer is a metastatic breast cancer.

Methods of identifying whether a patient has a breast cancer is locally advanced or metastatic are well known in the art and can be readily used by the skilled person. In another embodiment, the locally advanced or metastatic breast cancer overexpresses hormone receptors, such as estrogen receptors.

In another embodiment, the locally advanced or metastatic breast cancer is positive for estrogen receptor (ER) and/or progesterone receptor (PgR). Preferably the locally advanced or metastatic breast cancer is also negative for HER2. Also preferably the locally advanced or metastatic breast cancer is also refactory to non-steroidal aromatase inhibitor (e.g. letrozole and/or anastrozole).

Methods of identifying whether a patient has a breast cancer that is positive for estrogen receptor (ER) and/or progesterone receptor (PgR), and whether it overexpresses hormone receptors, such as estrogen receptor, are well known in the art.

In another embodiment, the patient to be treated has locally advanced or metastatic breast cancer not deemed amenable to curative surgery or curative radiation therapy.

In another embodiment, the patient to be treated is a postmenopausal woman.

In another embodiment, the patient to be treated shows objective evidence of recurrence or progressive disease on or after the last line of systemic therapy for breast cancer prior to study entry.

In another embodiment, the patient to be treated has a measurable lesion according to RECIST version 1.1 or Bone lesions only: lytic or mixed (lytic+sclerotic) in the absence of measurable lesion as defined above.

In another embodiment, the patient to be treated has a Eastern Cooperative Oncology Group performance score <=2.

In another embodiment, the patient to be treated has a life expectancy of >=6 months in the opinion of the investigator.

In another embodiment, the patient to be treated has fasting plasma glucose <8.9 mmol/L (<160 mg/dL) and HbA1c<8.0%.

In another embodiment the patient to be treated has not been treated with agents targeting on IGF pathway, phosphoinositide 3-kinase (PI3K) signaling pathway, protein kinase B (AKT), or mammalian target of rapamycin (mTOR) pathways.

In another embodiment the patient to be treated has not been treated with exemestane.

In another embodiment the patient to be treated does not have known hypersensitivity to monoclonal antibody, mTOR inhibitors (e.g. sirolimus), or to the excipients of any study drugs.

In another embodiment the patient to be treated does not have ovarian suppression by ovarian radiation or treatment with a luteinizing hormone-releasing hormone (LH-RH) agonist.

In another embodiment the patient to be treated has not less than one week after receiving immunization with attenuated live vaccines prior to treatment.

In another embodiment the patient to be treated has not received radiotherapy within 4 weeks prior to run-in treatment, except in case of localized radiotherapy for analgesic purpose or for lytic lesions at risk of fracture which can then be completed within two weeks prior to study treatment

In another embodiment the patient to be treated has not received chemotherapy, biological therapy (other than bevacizumab), immunotherapy or investigational agents within 5 half-life of the drug or within two weeks prior to the start of treatment, whichever is longer; bevacizumab treatment within 4 weeks prior to start of study treatment.

In another embodiment the patient to be treated has not received hormonal treatment for breast cancer within 2 weeks prior to start of treatment.

In another embodiment the patient to be treated has not received major surgery within 4 weeks before starting treatment or scheduled for surgery during the projected course of the treatment.

In another embodiment the patient to be treated is not receiving concomitant immunosuppressive agents or chronic corticosteroids use except Topical applications, inhaled sprays, eye drops or local injections or on stable low dose of corticosteroids for at least two weeks before study treatment.

In another embodiment the patient to be treated does not have chronic hepatitis B infection, chronic hepatitis C infection and/or is a known HIV carrier.

In another embodiment the patient to be treated does not show QTcF prolongation >470 ms or QT prolongation deemed clinically relevant.

In another embodiment the patient to be treated does not show disease that is rapidly progressing or life threatening such as extensive symptomatic visceral disease including hepatic involvement and pulmonary lymphangitic spread of tumor.

In another embodiment the patient to be treated does not have history of brain or other CNS metastases.

In another embodiment the patient to be treated does not have bilateral diffuse lymphangitic carcinomatosis.

In another embodiment the patient to be treated does not have hypokalemia of Grade >1.

In another embodiment the patient to be treated does not have history of another primary malignancy within 5 years, with the exception of adequately treated in-situ carcinoma of the cervix, uteri, basal or squamous cell carcinoma or non-melanomatous skin cancer.

In another embodiment the patient to be treated does not have family history of long QT syndrome.

In another embodiment the patient to be treated does not have any concomitant serious illness or organ system dysfunction which would either compromise patient safety or interfere with the of the safety and anti-tumor activity of the medicaments.

In another embodiment the patient to be treated is not being treated with drugs recognized being strong or moderate CYP3A4 and/or PgP inhibitors and/or strong CYP3A4 inducers within 2 weeks prior to treatment.

In another embodiment the patient to be treated has not received more than two lines of chemotherapy for locally advanced or metastatic breast cancer.

An IGF receptor antagonist within the context of the invention is a compound that interferes with, either directly or indirectly, and reduces or blocks IGF receptor signaling. Preferably, an IGF receptor antagonist is a compound that reduces or blocks binding of IGF ligand to its receptor, or inhibits the tyrosine kinase activity of the IGF receptor.

In a further embodiment, the IGF receptor antagonist of the present invention is an antibody that binds to IGF ligand and thus reduces or prevents binding of the ligand to the receptor. In another embodiment, the IGF receptor antagonist is an antibody that binds to the IGF-1 receptor and thus reduces or prevents binding of the ligand to the receptor. By blocking receptor-ligand binding, ligand-induced receptor signaling through the tyrosine kinase activity of the receptor is reduced or prevented. Such antibodies are generally referred to as neutralizing antibodies. In another aspect, the present invention pertains to an IGF receptor antagonist that neutralizes the growth promoting properties of the insulin-like growth factors, IGF-1 and IGF-2.

The term “antibody” encompasses antibodies, antibody fragments, antibody-like molecules and conjugates with any of the above. Antibodies include, but are not limited to, poly- or monoclonal, chimeric, humanized, human, mono-, bi- or multispecific antibodies. The term “antibody” shall encompass complete immunoglobulins as they are produced by lymphocytes and for example present in blood sera, monoclonal antibodies secreted by hybridoma cell lines, polypeptides produced by recombinant expression in host cells, which have the binding specificity of immunoglobulins or monoclonal antibodies, and molecules which have been derived from such immunoglobulins, monoclonal antibodies, or polypeptides by further processing while retaining their binding specificity. In particular, the term “antibody” includes complete immunoglobulins comprising two heavy chains and two light chains. In another embodiment, the term encompasses a fragment of an immunoglobulin, like Fab fragments. In another embodiment, the term “antibody” encompasses a polypeptide having one or more variable domains derived from an immunobulin, like single chain antibodies (scFv), single domain antibodies, and the like.

In a further embodiment, the IGF receptor antagonist of the invention is an antibody against IGF-1, an antibody against IGF-2, an antibody binding both IGF-1 and IGF-2, an antibody against IGF-1 receptor (IGF-1R), or an inhibitor of IGF-1R tyrosine kinase activity.

In another embodiment, the IGF receptor antagonist is an IGF ligand antibody having heavy chain complementary determining regions of SEQ ID NO: 1 (HCDR1), SEQ ID NO: 2 (HCDR2), and SEQ ID NO: 3 (HCDR3) and light chain determining regions of SEQ ID NO: 4 (LCDR1), SEQ ID NO: 5 (LCDR2), and SEQ ID NO: 6 (LCDR3).

In another embodiment, the IGF receptor antagonist is an IGF ligand antibody having heavy chain complementary determining regions of SEQ ID NO: 11 (HCDR1), SEQ ID NO: 12 (HCDR2), and SEQ ID NO: 13 (HCDR3) and light chain determining regions of SEQ ID NO: 14 (LCDR1), SEQ ID NO: 15 (LCDR2), and SEQ ID NO: 16 (LCDR3).

In another embodiment, the IGF receptor antagonist is an IGF ligand antibody having heavy chain complementary determining regions of SEQ ID NO: 21 (HCDR1), SEQ ID NO: 22 (HCDR2), and SEQ ID NO: 23 (HCDR3) and light chain determining regions of SEQ ID NO: 24 (LCDR1), SEQ ID NO: 25 (LCDR2), and SEQ ID NO: 26 (LCDR3).

In another embodiment, the IGF receptor antagonist is an IGF ligand antibody having heavy chain complementary determining regions of SEQ ID NO: 31 (HCDR1), SEQ ID NO: 32 (HCDR2), and SEQ ID NO: 33 (HCDR3) and light chain determining regions of SEQ ID NO: 34 (LCDR1), SEQ ID NO: 35 (LCDR2), and SEQ ID NO: 36 (LCDR3).

In another embodiment, the IGF receptor antagonist is an IGF ligand antibody having a heavy chain variable region of SEQ ID NO: 7 and a light chain variable region of SEQ ID NO: 8.

In another embodiment, the IGF receptor antagonist is an IGF ligand antibody having a heavy chain variable region of SEQ ID NO: 17 and a light chain variable region of SEQ ID NO: 18.

In another embodiment, the IGF receptor antagonist is an IGF ligand antibody having a heavy chain variable region of SEQ ID NO: 27 and a light chain variable region of SEQ ID NO: 28.

In another embodiment, the IGF receptor antagonist is an IGF ligand antibody having a heavy chain variable region of SEQ ID NO: 37 and a light chain variable region of SEQ ID NO: 38.

In another embodiment, the IGF receptor antagonist is an IGF ligand antibody having a heavy chain variable region of SEQ ID NO: 41 and a light chain variable region of SEQ ID NO: 42.

In another embodiment, the IGF receptor antagonist is an IGF ligand antibody having a heavy chain variable region of SEQ ID NO: 43 and a light chain variable region of SEQ ID NO: 44.

In another embodiment, the IGF receptor antagonist is an IGF ligand antibody having a heavy chain of SEQ ID NO: 9, and a light chain of SEQ ID NO: 10.

In another embodiment, the IGF receptor antagonist is an IGF ligand antibody having a heavy chain of SEQ ID NO: 19, and a light chain of SEQ ID NO: 20.

In another embodiment, the IGF receptor antagonist is an IGF ligand antibody having a heavy chain of SEQ ID NO: 29, and a light chain of SEQ ID NO: 30.

In another embodiment, the IGF receptor antagonist is an IGF ligand antibody having a heavy chain of SEQ ID NO: 39, and a light chain of SEQ ID NO: 40.

In another embodiment, the IGF receptor antagonist is an IGF receptor antibody having a heavy chain of SEQ ID NO: 45, and a light chain of SEQ ID NO: 46.

Preferably the IGF receptor antagonist is 60833, an antibody against IGF ligand having a heavy chain of SEQ ID NO: 39 and a light chain of SEQ ID NO: 40. Its manufacture has been disclosed in WO 2010/066868.

In another embodiment, the IGF receptor antagonist is dusigitumab, figitumumab, dalotuzumab, cixutumumab, robatumumab, or ganitumab.

In another embodiment, the IGF receptor antagonist is linsitinib.

Manufacture and therapeutic use of the aforementioned antibodies is disclosed in the art and are well known to the skilled person, and specific disclosures can be identified in WO2002/53596, WO2007/070432, WO2008/152422, WO2008/155387, and WO2010/066868.

In one embodiment, the antibody is produced by recombinant expression in a mammalian host cell, purified by a series of chromatographic and non-chromatographic steps, and formulated in an aqueous buffer composition for parenteral (intravenous) infusion or injection at an antibody concentration of 10 mg/ml, said buffer comprising for example 25 mM Na citrate pH 6, 115 mM NaCl, and 0.02% polysorbate 20. For intravenous infusion, the pharmaceutical composition may be diluted with a physiological solution, e.g. with 0.9% sodium chloride or G5 solution.

The antibody may be administered to the patient at a dose between 1 mg/kg to 20 mg/kg, by one or more separate administrations, or by continuous infusion, e.g. infusion over 1 hour. A typical treatment schedule usually involves administration of the antibody once every week to once every three weeks. For example, a weekly dose could be 5, 10, or 15 mg/kg.

The antibody may also be administered to the patient at a dose of between 500 and 1000 mg per week, optionally 750 or 1000 mg per week.

The IGF receptor antagonist is administered to the patient in combination with administration of exemestane and everolimus. “In combination” means that the drugs are administered to the same patient within a certain time frame to achieve a therapeutic effect caused by the combined effects of modes of action. In one aspect, exemestane and everolimus are administered on the same day as the IGF receptor antagonist. In another aspect of the invention, exemestane and everolimus are administered one, two, three, four, five, six or seven days before or after administration of the IGF receptor antagonist.

In another embodiment, all three active compounds are present within the same pharmaceutical composition. Hence, in another embodiment, the invention pertains to a pharmaceutical composition, comprising an IGF receptor antagonist and exemestane and everolimus, together with a pharmaceutically acceptable carrier.

Exemestane is a member of the class of drugs known as aromatase inhibitors. Some breast cancers require estrogen to grow. Those cancers have estrogen receptors (ERs), and are called ER-positive. They may also be called estrogen-responsive, hormonally-responsive, or hormone-receptor-positive. Aromatase is an enzyme that synthesizes estrogen. Aromatase inhibitors block the synthesis of estrogen. This lowers the estrogen level, and slows the growth of cancers.

The structure of exemestane is provided below

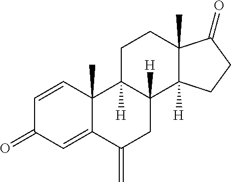

The systematic name is 6-Methylideneandrosta-1,4-diene-3,17-dione

Exemestane can be obtained commerically under the trade name Aromasin. Manufacture, formulation, and use of the exemestane can be found in the state of the art.

Preferably exemestane will be supplied to patients orally at a dosage of 25 mg per day;

Everolimus is an inhibitor of mammalian target of rapamycin (mTOR).

The structure of everolimus is provided below

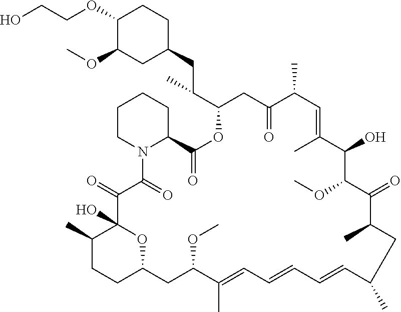 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OCCO)[C@H](OC)C2)CC(=O)[C@H](C)/C=C(\C)[C@@H](O)[C@@H](OC)C(=O)[C@H](C)C[C@H](C)/C=C/C=C/C=C/1C]

The systematic name is dihydroxy-12-[(2R)-1-[(1S,3R,4R)-4-(2-hydroxyethoxy)-3-methoxycyclohexyl]propan-2-yl]-19,30-dimethoxy-15,17,21,23,29,35-hexamethyl-11,36-dioxa-4-azatricyclo[30.3.1.0 hexatriaconta-16,24,26,28-tetraene-2,3,10,14,20-pentone

Everolimus can be obtained commerically under the trade name Afinitor. Manufacture, formulation, and use of the exemestane can be found in the state of the art.

Preferably everolimus will be supplied to patients orally at a dosage of between 5 mg and 10 mg per day; optionally 7.5 mg per day;

In another embodiment, the invention relates to a method of treatment of breast cancer comprising administering a therapeutically effective amount of an IGF receptor antagonist to a patient in need thereof, and additionally administering a therapeutically effective amount of an exemestane and everolimus to the same patient.

Preferably the breast cancer is locally advanced or metastatic breast cancer.

A “therapeutically effective amount” of the IGF receptor antagonist or exemestane and everolimus to be administered is the minimum amount necessary to prevent, ameliorate, or treat a locally advanced or metastatic breast cancer.

### Example 1

### Study of IGF Ab 60833 in Combination with Exemestane and Everolimus Versus Exemestane and Everolimus Alone in Women with Locally Advanced or Metastatic Breast Cancer

### Introduction

The study proposed here investigates the effect of IGF Ab 60833 in combination with Exemestane and Everolimus in in estrogen receptor positive metastatic breast cancer.

In more detail, the phase I part will determine the Maximum Tolerated Dose (MTD) and Recommended Phase II Dose (RP2D) of IGF Ab 60833 and everolimus in combination with exemestane in women with HR+/HER2− advanced breast cancer. The Phase II part will evaluate the antitumor activity of IGF Ab 60833 in combination with exemestane and everolimus compared to exemestane and everolimus alone in women with HR+/HER2− advanced breast cancer.

### Background

IGF Ab 60833 is a fully human monoclonal antibody (HumAb) of the IgG1 isotype. The Ab binds with high affinity to IGF-1 and IGF-2, and potently neutralizes the proliferative and prosurvival cellular signaling triggered by both proteins.

Everolimus is a selective mTOR (mammalian target of rapamycin) inhibitor.

Exemestane is an oral steroidal aromatase inhibitor that is used in ER-positive breast cancer in addition to surgery and/or radiation in post-menopausal women.

### Administration

IGF Ab 60833 will be supplied to the study sites as a concentrate for solution for injection/infusion. A total of 1000 mg milligram(s) will be supplied to patients intravenously. The patient will have continuous treatment until disease progression, intolerable AEs, consent withdrawal or non-compliance with the study.

Everolimus will be supplied to patients orally at a dosage of 10 mg milligram per day. The patient will have continuous treatment until disease progression, intolerable AEs, consent withdrawal or non-compliance with the study.

Exemestane will also be supplied to patients orally.

### Medical Condition or Disease Under Investigation

The study proposed here will investigate Locally Advanced or Metastatic Breast Cancer positive for estrogenreceptor (ER) and/or progesterone receptor (PgR) and negative for HER2 which is refractory to non-steroidal aromatase inhibitor (letrozole and/or anastrozole).

### Principle Inclusion Criteria

The following criteria will be used to evaluate the inclusion of patients in to the study.

Histologically-confirmed locally advanced (aBC) or metastatic breast cancer (mBC) not deemed amenable to curative surgery or curative radiation therapy

Tumors are positive for estrogen-receptor (ER) and/or progesterone receptor (PgR).

Tumors must be negative for HER2 per local lab testing.

Must have adequate archival tumor tissue from surgery or biopsy.

Postmenopausal women.

Objective evidence of recurrence or progressive disease on or after the last line of systemic therapy for breast cancer prior to study entry

The patient is disease refractory to non-steroidal aromatase inhibitor (letrozole and/or anastrozole)

Patients must have Measurable lesion according to RECIST version 1.1 or Bone lesions only: lytic or mixed (lytic+sclerotic) in the absence of measurable lesion as defined above

Eastern Cooperative Oncology Group performance score <=2.

Life expectancy of >=6 months in the opinion of the investigator

Fasting plasma glucose <8.9 mmol/L (<160 mg/dL) and HbA1c<8.0%

Adequate organ function

Recovered from any previous therapy related toxicity to <=Grade 1 at study entry (except for stable sensory neuropathy <=Grade 2 and alopecia)

Written informed consent that is consistent with ICH-GCP guidelines and local regulations Inclusion criteria for the biopsy substudy are identical to the main study of the phase II part except for the following two inclusion criteria:

Fresh tumor biopsy should be taken when deemed safe and feasible by the investigator and upon informed consent by the patient. Bone lesion is not recommended for biopsy

Patients eligible to undergo tumor biopsy should have normal coagulation parameters (INR and PTT within normal range)

### Principle Exclusion Criteria

The following criteria will be used to evaluate the exclusion of patients in to the study.

Previous treatment with agents targeting on IGF pathway, phosphoinositide 3-kinase (PI3K) signaling pathway, protein kinase B (AKT), or mammalian target of rapamycin (mTOR) pathways

Prior treatment with exemestane

Known hypersensitivity to monoclonal antibody, mTOR inhibitors (e.g. sirolimus), or to the excipients of any study drugs

Ovarian suppression by ovarian radiation or treatment with a luteinizing hormone-releasing hormone (LH-RH) agonist

Less than one week after receiving immunization with attenuated live vaccines prior to study treatment

Radiotherapy within 4 weeks prior to run-in treatment, except in case of localized radiotherapy for analgesic purpose or for lytic lesions at risk of fracture which can then be completed within two weeks prior to study treatment

Chemotherapy, biological therapy (other than bevacizumab), immunotherapy or investigational agents within 5 half-life of the drug or within two weeks prior to the start of study treatment, whichever is longer; bevacizumab treatment within 4 weeks prior to start of study treatment

Hormonal treatment for breast cancer within 2 weeks prior to start of study treatment

Major surgery in the judgement of the investigator within 4 weeks before starting study treatment or scheduled for surgery during the projected course of the study

Patients receiving concomitant immunosuppressive agents or chronic corticosteroids use except Topical applications, inhaled sprays, eye drops or local injections or Patients on stable low dose of corticosteroids for at least two weeks before study entry

Chronic hepatitis B infection, chronic hepatitis C infection and/or known HIV carrier

QTcF prolongation >470 ms or QT prolongation deemed clinically relevant by the investigator

Disease that is considered by the investigator to be rapidly progressing or life threatening such as extensive symptomatic visceral disease including hepatic involvement and pulmonary lymphangitic spread of tumor

History of brain or other CNS metastases

Bilateral diffuse lymphangitic carcinomatosis

Hypokalemia of Grade >1

History of another primary malignancy within 5 years, with the exception of adequately treated in-situ carcinoma of the cervix, uteri, basal or squamous cell carcinoma or non-melanomatous skin cancer

Family history of long QT syndrome

Any concomitant serious illness or organ system dysfunction which in the opinion of the investigator would either compromise patient safety or interfere with the evaluation of the safety and anti-tumor activity of the test drug(s)

Patients being treated with drugs recognized being strong or moderate CYP3A4 and/or PgP inhibitors and/or strong CYP3A4 inducers within 2 weeks prior to study entry

Patients received more than two lines of chemotherapy for locally advanced or metastatic breast cancer (For the Phase II: more than one line)

### End Points

The primary end points are:

1: Progression-free survival (PFS), the timepoint of evaluation being up to 10.8 months.

2: occurrence of Dose Limiting Toxicity (DLT)—phase I part, the timepoint of evaluation being up to 28 days.

The secondary End Points are:

1: Time to progression (TTP), defined as the duration of time from the date of C1V1 until the date of the first objective tumor progression, the timepoint of evaluation being up to 10.8 months.

2: Objective response (OR), defined as complete response (CR) or partial response (PR) (CR+PR), the timepoint of evaluation being up to 10.8 months.

3: Time to objective response, the timepoint of evaluation being up to 10.8 months.

4: Duration of objective response, the timepoint of evaluation being up to 10.8 months.

5: Clinical benefit (CB), defined as best overall response of complete response (CR) or partial response (PR), or stable disease (SD) >=6 months, or Non-CR/Non-PD for >=6 months (CR+PR+SD6m+Non-CR/Non-PD6m), the timepoint of evaluation being up to 10.8 months.

6: Duration of clinical benefit, the timepoint of evaluation being up to 10.8 months.

### Example 2

### Preliminary study of IGF Ab 60833 in Combination with Exemestane and Everolimus Versus Exemestane and Everolimus Alone in Women with Locally Advanced or Metastatic Breast Cancer

Preliminary investigations were performed in to the effect of a triple combination of IGF Ab 60833, everolimus and exemestane, against a double combination of everolimus and exemestane. The investigation was conducted in ER positive breast cancer cell line engineered to expression the human aromatase gene.

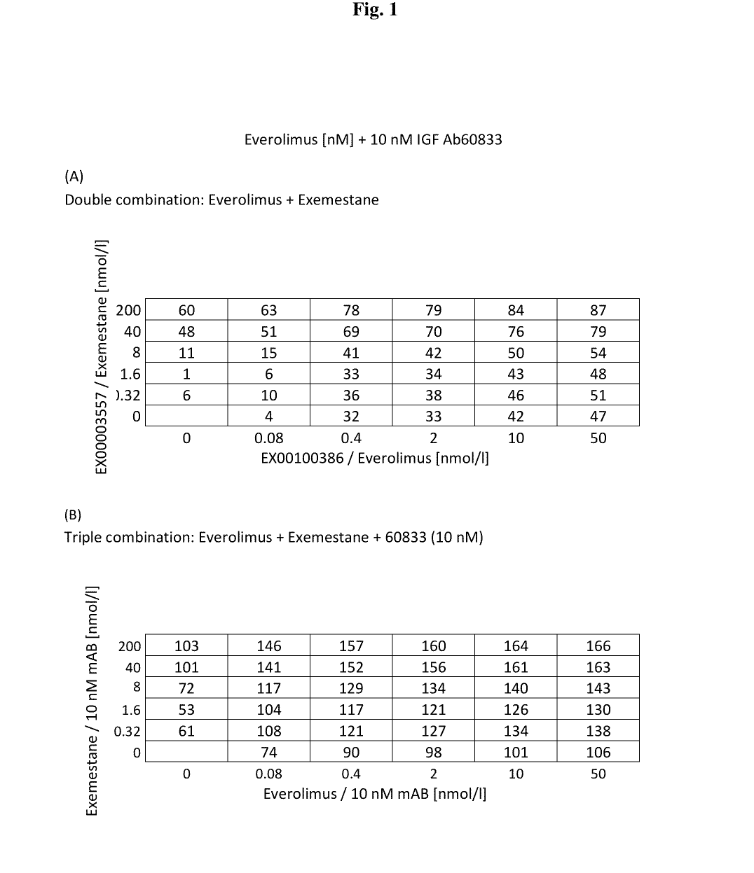

FIG. 1 shows a comparison of the double combination of everolimus and exemestane (top panel) against the triple combination of IGF Ab 60833, everolimus and exemestane (bottom panel).

It can be clearly seen from a comparison of the two panels that IGF Ab 60833 causes a surprisingly large increase in cell growth inhibition to the everolimus and exemestane combination.

### Example 3

### Effect of IGF Ab 60833 in Combination with the mTOR Inhibitor Everolimus and the Aromatase Inhibitor Exemestane on Growth and Biomarker Phosphorylation Status of the Breast Cancer Cell Line MCF7aro

### Summary

The aim of the present study was to explore the in vitro effect of the combination of IGF Ab 60833, a fully human antibody that binds to IGF-1 and IGF-2, with the mTORC1 inhibitor everolimus and the aromatase inhibitor exemestane on the proliferation of MCF7aro cells, derived from the estrogen receptor positive breast cancer cell line MCF7, engineered to stably express the human aromatase protein.

MCF7aro breast cancer cells were cultured in steroid-deprived medium, supplemented with the estradiol precursor androstenedione and incubated with IGF Ab 60833, everolimus and exemestane as single agents or in combination, to determine effects on PI3K/mTOR pathway signaling, cell proliferation and survival.

Whereas treatment of MCF7aro cells with everolimus alone resulted in enhanced AKT phosphorylation, co-treatment with IGF Ab 60833 prevented the increase in phospho AKT and led to more pronounced inhibition of downstream signaling. The triple combination of IGF Ab 60833 with everolimus and exemestane resulted in enhanced cell growth inhibition and induction of apoptosis as compared to treatment with the dual combination of everolimus and exemestane.

This study has demonstrated that addition of IGF Ab 60833 to the combination of everolimus and exemestane, which is the current standard-of-care treatment in hormone receptor positive breast cancer, leads to improved anti-neoplastic activity in vitro.

### Introduction

Both the insulin-like growth factor (IGF) signalling system as well as the PI3 kinase/mTOR signaling pathway plays an important role in the regulation of survival and proliferation of mammalian cells.

The insulin-like growth factor (IGF) signalling system consists of ligands (insulin-like growth factors 1 and 2 (IGF-1, IGF-2), IGF-binding proteins (IGFBPs), and receptors (insulin-like growth factor 1 receptor (IGF-1R), IGF-2R, and insulin receptor (IR). Evidence that targeting IGF may be useful in cancer treatment was first recognised decades ago. Research in IGF signalling has shown that it controls key cellular activities, including proliferation, growth, and survival and is often deregulated in neoplasia.

Expression of IGF-IR has been shown to be increased in a variety of cancers, including lung, colon, prostate, breast, ovarian, liver cancer, and sarcomas. The IGF system has thus become a target for anti-cancer drug development. Pharmacologic targeting strategies include inhibition of receptor function with IGF-1R antibodies or small molecule receptor tyrosine kinase inhibitors. IGF Ab 60833 offers an alternative approach by acting as an IGF ligand neutralizing antibody. In addition to expression of the IGF-1R, there is evidence for autocrine and/or paracrine expression of the IGF ligands, particularly autocrine IGF-2, in multiple cancers. IGF-2 has also been shown to bind and signal through the insulin receptor isoform A (IR-A) which is expressed on foetal and cancer cells. Thus, the strategy of targeting IGF-1 and IGF-2 has the potential for therapeutic advance.

IGF Ab 60833 is a fully human monoclonal antibody which binds IGF-1 and IGF-2 and neutralizes their biological effects by blocking the interaction with their cognate receptors, with the potential of anti-neoplastic activity.

A large number of pre-clinical studies have evaluated the functional role of the serine/threonine kinase mTOR and other components of the PI3 kinase/mTOR pathway in cancer. Activation of the PI3 kinase/mTOR signalling pathway by mutation or amplification of pathway components has been found in a large proportion of cancers of different origin, suggesting an important role of pathway hyperactivation in the aetiology of the disease.

The mTOR kinase functions in two cellular multi-protein complexes, mTORC1 and mTORC2, with distinct substrates and mechanisms of activation, and regulates survival, growth and cell cycle progression of cells.

Hyperactivation of the PI3K/mTOR pathway has been claimed to result in resistance to standard therapy, e.g. to endocrine therapy in hormone receptor positive breast cancer.

Inhibition of mTOR, in combination with other agents, is therefore considered to be an attractive approach to cancer therapy.

Rapamycin derivatives (rapalogs) have been approved for the treatment of several cancer types. They act as allosteric inhibitors of the mTORC1 protein complex. The rapalog everolimus (Afinitor®, Novartis Pharma), in combination with endocrine therapy, i.e. the aromatase inhibitor exemestane (Aromasin®, Pfizer Inc.), has been approved for the treatment of estrogen receptor (ER)-positive breast cancer.

Despite the fact that rapamycin analogues such as everolimus have shown clinical activity in several cancers, pre-clinical and clinical data suggest that, when mTORC1 is blocked, there may be acquired resistance by release of a negative feedback loop, resulting in induction of AKT phosphorylation, i.e. re-activation of PI3 kinase/mTOR pathway signaling.

In a preclinical model of Ewing's sarcoma, improved anti-tumor efficacy was observed when rapamycin was combined with IGF Ab 60833. IGF Ab 60833 was shown to inhibit the rapamycin induced increase in pAKT indicating that the elevated pAKT level was due to enhanced IGF ligand driven signalling.

Together, preclinical data as well as clinical evidence indicate that combination of agents targeting more than one pathway component, known as vertical pathway inhibition, results in a more pronounced blockade and improved anti-cancer efficacy.

### Objectives

The aim of the present study was to explore the in vitro effect of the combination of IGF Ab 60833, with the mTORC1 inhibitor everolimus and the aromatase inhibitor exemestane on the proliferation of MCF7aro cells, derived from the estrogen receptor positive breast cancer cell line MCF7, engineered to stably express the human aromatase protein.

### Study Design

Three independent experiments were conducted to determine the anti-proliferative effect of IGF Ab 60833 in combination with everolimus and exemestane in MCF7aro breast cancer cells. Cell proliferation and viability following 6 days of incubation with test compounds was determined using an assay that monitors the reducing environment of the living cells in each assay well.

The effect on PI3 kinase/mTOR pathway signalling was assessed by Western Blot analysis, induction of apoptosis was monitored using the Meso Scale Discovery Apoptosis Whole Cell Lysate Kit.

IGF Ab 60833 comprises a heavy chain of SEQ ID NO: 39 and a light chain of SEQ ID NO: 40. Its manufacture has been disclosed in WO 2010/066868.

Everolimus (EX00100386) as used in this study has the chemical structure provided in herein.

Exemestane (EX0003557) as used in this study has the chemical structure provided in herein.

MCF7aro cells stably express the human aromatase protein. They were derived from MCF7 human ER-positive breast adenocarcinoma cells (ATCC, HTB-22), by transfection of aromatase cDNA, Geneticin selection (neomycin) and clonal purification.

### Methods

### Cell Culture

MCF7aro cells were cultivated in MEM growth medium supplemented with 10% FBS, 2 mM GlutaMAX, 1 mM sodium pyruvate and 0.1 mg/ml Geneticin as monolayer cultures. The cells were maintained in 175 cm² tissue culture flasks at 37° C. and 5% CO₂ in a humidified atmosphere.

For cell proliferation assays and Western Blot analysis the cells were steroid-deprived for 72 h by cultivation in starvation medium (MEM alpha without phenol red, supplemented with 10% charcoal stripped FBS, 2 mM GlutaMAX and 1 mM sodium pyruvate).

### Cell Proliferation Assay of Adherently Growing MCF7aro Breast Cancer Cells

This assay was used to determine the inhibitory effect of IGF Ab 60833, everolimus and exemestane on the viability and growth of MCF7aro cells.

### Assay Conditions:

Adherent cells were detached with Trypsin/EDTA solution, resuspended in starvation medium and diluted to 25,000 cells per ml in starvation medium. 200 μl cell suspension (5,000 cells) per well were plated in four sterile NuncTm Edge 96-well plates (except wells B1/B2: medium control, add only 200 μl starvation medium). Plates were incubated for 48 h in a humidified incubator at 37° C. and 5% CO₂. Two days later supernatants were aspirated and 150 μl assay medium (MEM alpha, without phenol red, supplemented with 10% charcoal stripped FBS, 2 mM GlutaMAX, 1 mM sodium pyruvate and 1 nM androstenedione) was added to each well in each plate.

### Addition of Test Compounds:

For the combination of everolimus with exemestane, 50 μl/well of assay medium was added to all wells. 5-fold serial dilutions of everolimus (highest test concentration 50 nM), exemestane (highest test concentration 200 nM) or DMSO (vehicle-treated cell control and medium control) were added to the cells using the HP D300 Digital Dispenser.

For the triple combination of everolimus, exemestane and IGF Ab 60833, 40 nM of IGF Ab 60833 solution was prepared in assay medium. 50 μl/well of IGF Ab 60833 solution was added to the cells to yield a final test concentration of 10 nM. 50 μl/well assay medium was added to cell control and medium control wells. 5-fold serial dilutions of everolimus (highest test concentration 50 nM) and exemestane (highest test concentration 200 nM), or DMSO were added to the cells using the HP D300 Digital Dispenser. The final volume per well was 200 μl.

The final concentration of the solvent DMSO in the test wells was 0.1%. Everolimus and exemestane were tested at 5 concentrations, each measured in duplicate wells, as single agents or in combination.

After 2 days of incubation with test compounds in a humidified incubator at 37° C. and 5% CO₂, medium was changed to fresh assay medium and compounds were added again. After 6 days of total incubation time with test compounds, cells were stained with 20 μl of AlamarBlue® Cell Viability Reagent to assess cell viability. The plates were incubated for 6 hours at 37° C. and 5% CO₂ to allow cells to convert resazurin to resorufin. Total fluorescence intensity of each well was then measured in a Wallac VICTOR Multilabel Counter using an excitation wavelength of 544 nm and measuring emission at 590 nm.

At the time of test compound addition, “time zero” (t=0) untreated cell plates were stained with AlamarBlue® Cell Viability Reagent. 50 μl_{—} of assay medium and 20 μl of AlamarBlue® Cell Viability Reagent were added to each well containing cells in 150 μl assay medium. After incubation for 6 hours at 37° C. and 5% CO₂, total fluorescence intensity was measured.

The AlamarBlue® assay is designed to measure quantitatively the viability of cells by incorporating a fluorometric/colorimetric growth indicator based on the detection of metabolic activity. The fluorescent signal is proportional to total number of viable cells

When cells are alive they maintain a reducing environment within the cytosol. Resazurin, the active ingredient of the AlamarBlue® Cell Viability Reagent, is a non-toxic, cell permeable compound that is blue in colour and virtually non-fluorescent. Upon entering cells, resazurin is reduced to resorufin, which is red in colour and highly fluorescent. Viable cells continuously convert resazurin to resorufin, increasing the overall fluorescence and colour of the media surrounding cells.

### Data Analysis:

The AlamarBlue® assay output for vehicle-treated control cells after 6 days of incubation, corresponding to 100% cell viability, was taken as the reference signal for all subsequent calculations. Relative cell viability in compound-treated cultures (signal percent of control, “POC”) was calculated according to the following formula: POC (t=144 h)=100*fluorescence (compound wells)/fluorescence (control wells). In addition, for each compound-treated culture, the fluorescence signal after incubation for 144 hours (POC (t=144 h)) was related to the signal at the start of treatment (POC (t=0 h)): POC (t=0 h)=100*fluorescence at t=0 (control wells)/fluorescence at t=144 h (control wells).

To calculate concentration-response curves, the POC data were analyzed using a four-parameter log-logistic function without any upper or lower limitation. Relative cell growth inhibition (CGI %) in compound-treated cultures is calculated according to the following formula:

$$\% {CGI}^{144 h} = \{ \begin{matrix}{S}_{t}^{144} \geq {S}_{c}^{0} \text{:} [ 1 - \frac{{S}_{t}^{144 h} - {S}_{c}^{0 h}}{{S}_{c}^{144 h} - {S}_{c}^{0 h}} ] \times 100 \% \\ {S}_{t}^{144 h} < {S}_{c}^{0} \text{:} [ 1 - \frac{{S}_{t}^{144 h} - {S}_{c}^{0 h}}{{S}_{c}^{0 h}} ] \times 100 \%\end{matrix} \\ {S}_{t}^{144} = {POC}_{( t = 144 h )} \\ {S}_{c}^{0} = {POC}_{( t = 0 h )}$$

A CGI of >0% and <100% reflects a partial growth-inhibitory effect relative to vehicle-treated controls, a CGI of 100% is equivalent of complete blockade of growth, and a CGI of >100% is indicative of net cell death.

Results are evaluated by a CGI matrix, plotting multiple compound concentrations of test compound 1 against different concentrations of test compound 2.

### Preparation of Cell Lysates and Western Blot (Assessment of AKT and S6 Phosphorylation Status)

1.8×10⁶ MCF7aro cells were plated in 10 cm dishes in starvation medium (MEM alpha without phenol red, supplemented with 10% charcoal stripped FBS, 2 mM GlutaMAX and mM sodium pyruvate). After overnight incubation, medium was changed to assay medium (MEM alpha without phenol red, supplemented with 10% charcoal stripped FBS, 2 mM GlutaMAX, 1 mM sodium pyruvate and 1 nM androstenedione) and the cells were treated with 100 nM of IGF Ab 60833 or 0.32 nM of everolimus or a combination of antibody and mTORC1 inhibitor. As a control, cells were treated with vehicle (DMSO) only. At 24 h post treatment, the cells were lysed on ice with MSD Tris Lysis Buffer containing 20 mM Tris pH 7.5, 150 mM NaCl, 1 mM EDTA, 1 mM EGTA, 1% Triton X-100, completed with protease and phosphatase inhibitor cocktails. Prior to use, freshly prepared 2 mM PMSF was added to the buffer.

Total protein was isolated and protein concentration was quantified by Bradford protein assay according to the manufacturer's instructions.

30 μg of total protein was separated on a 4-12% Bis-Tris precast gel and blotted on a PVDF membrane with the BIO-RAD Trans-Blot® Turbo™ Instrument.

Membranes were blocked for 1 h in 5% skim milk in 1×TBS/0.1% Tween 20 at room temperature and then probed overnight at 4° C. with antibodies against the following proteins: pAKT (S473), pAKT (T308), AKT, pS6 (S235/236), S6, and Actin, which served as loading control. Antibody dilutions were prepared in 5% skim milk. After washing and incubation with secondary antibody, the immunoblotted proteins were visualized using the ECL Western blotting detection reagent according to the manufacturer's instructions.

### Primary Antibodies:

rabbit anti phospho AKT (S473 (Cell Signaling #9271, 1:1000)
rabbit anti phospho AKT (T308) (Cell Signaling #2965, 1:1000)
rabbit anti phospho S6 Ribosomal Protein (S235/236) (Cell Signaling #2211, 1:2000)
rabbit anti AKT (Cell Signaling #9272, 1:2000)
rabbit anti S6 Ribosomal Protein (Cell Signaling #2217, 1:2000)
rabbit anti beta Actin (Dako #P0448, 1:1000)

### Secondary Antibody:

goat anti rabbit IgG, HRP conjugated (Dako #P0448, 1:1000)

### Preparation of Cell Lysates and Western Blot (Assessment of Cleaved PARP)

1.5×10⁶ MCF7aro cells were plated in 10 cm dishes in starvation medium (MEM alpha without phenol red, supplemented with 10% charcoal stripped FBS, 2 mM GlutaMAX and 1 mM sodium pyruvate). After overnight incubation, medium was changed to assay medium (MEM alpha without phenol red, supplemented with 10% charcoal stripped FBS, 2 mM GlutaMAX, 1 mM sodium pyruvate and 1 nM androstenedione) and the cells were treated with vehicle (DMSO), or 1 μM of IGF Ab 60833, or 1 μM exemestane, or 1 μM everolimus, or a combination of exemestane and everolimus, or a combination of all three inhibitors. At 72 h post treatment, the cells were lysed on ice with MSD Tris Lysis Buffer containing 20 mM Tris pH 7.5, 150 mM NaCl, 1 mM EDTA, 1 mM EGTA, 1% Triton X-100, completed with protease and phosphatase inhibitor cocktails. Prior to use, freshly prepared 2 mM PMSF was added to the buffer.

Total protein was isolated and protein concentration was quantified by Bradford protein assay according to the manufacturer's instructions.

20 μg of total protein was separated on a 4-12% Bis-Tris precast gel and blotted on a PVDF membrane with the BIO-RAD Trans-Blot® Turbo™ Instrument.

Membranes were blocked for 1 h in 5% skim milk in 1×TBS/0.1% Tween 20 at room temperature and then probed overnight at 4° C. with antibodies against PARP and Actin, which served as loading control. Antibody dilutions were prepared in 5% skim milk. After washing and incubation with secondary antibody, the immunoblotted proteins were visualized using the ECL Western blotting detection reagent according to the manufacturer's instructions.

### Primary Antibodies:

rabbit anti PARP (Cell Signaling #9542, 1:1000)
rabbit anti beta Actin (Cell Signaling #4967, 1:3000)
goat anti rabbit IgG, HRP conjugated

### Secondary Antibody:

goat anti rabbit IgG, HRP conjugated (Dako #P0448, 1:1000)

### Determination of Cleaved PARP in Cell Lysates by MSD Apoptosis Panel

Cells lysates were prepared according to the Meso Scale Discovery cell lysis protocol. 20 μg of clarified lysate was analyzed in duplicates with MSD Apoptosis Panel Whole Cell Lysate Kit, measuring cleaved PARP signals, according to the manufacturer's instructions. Plates were read on the SECTOR Imager 6000 plate reader.

### Results

### Effect on Cell Growth of MCF7aro Breast Cancer Cells

The inhibitory activity of everolimus, exemestane and IGF Ab 60833, as single agents or in combination, on the proliferation of MCF7aro cells was determined in monolayer cultures. For the combination of everolimus and exemestane, 5 concentrations of each drug were tested both as single agents and combined in a matrix format. For the triple combination, the same layout was applied for everolimus and exemestane, and IGF Ab 60833 was added at a fixed concentration of 10 nM to each sample. Three independent experiments with duplicate determinations were carried out for the dual and for the triple combination.

After 6 days of incubation, everolimus and exemestane as single agents showed a dose-dependent, partial reduction of cell growth with a maximal mean CGI of 47% and 60%, respectively. The combination of everolimus and exemestane was more effective, i.e. almost complete cell growth inhibition (mean CGI of >90%) was achieved at 6 different concentration ratios, with a maximal mean CGI of 103% at the highest concentration used for each inhibitor.

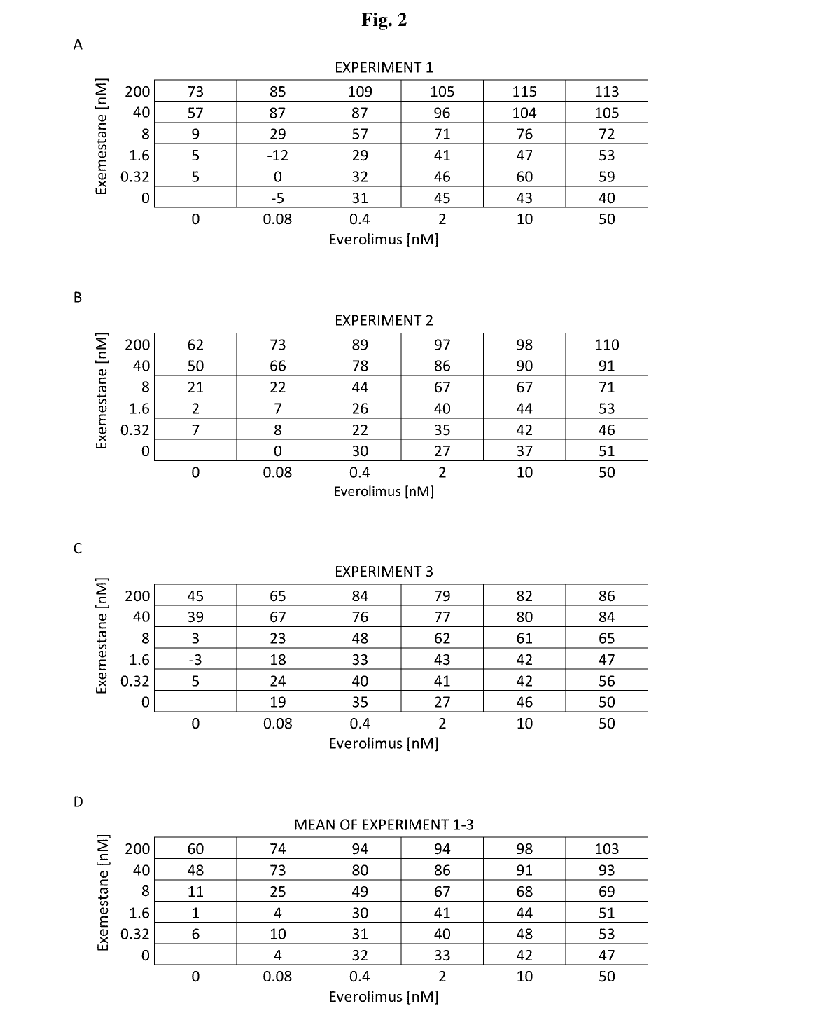

The data from the individual experiments and the mean CGI values are shown in FIG. 2.

Addition of a fixed concentration of 10 nM of IGF Ab 60833 to the everolimus/exemestane combination resulted in a further enhancement of the anti-proliferative effect. A pronounced induction of net cell death (mean CGI of >120%) in 13 different concentration ratios, with a maximal mean CGI of 149%, after 6 days of incubation with compounds was observed.

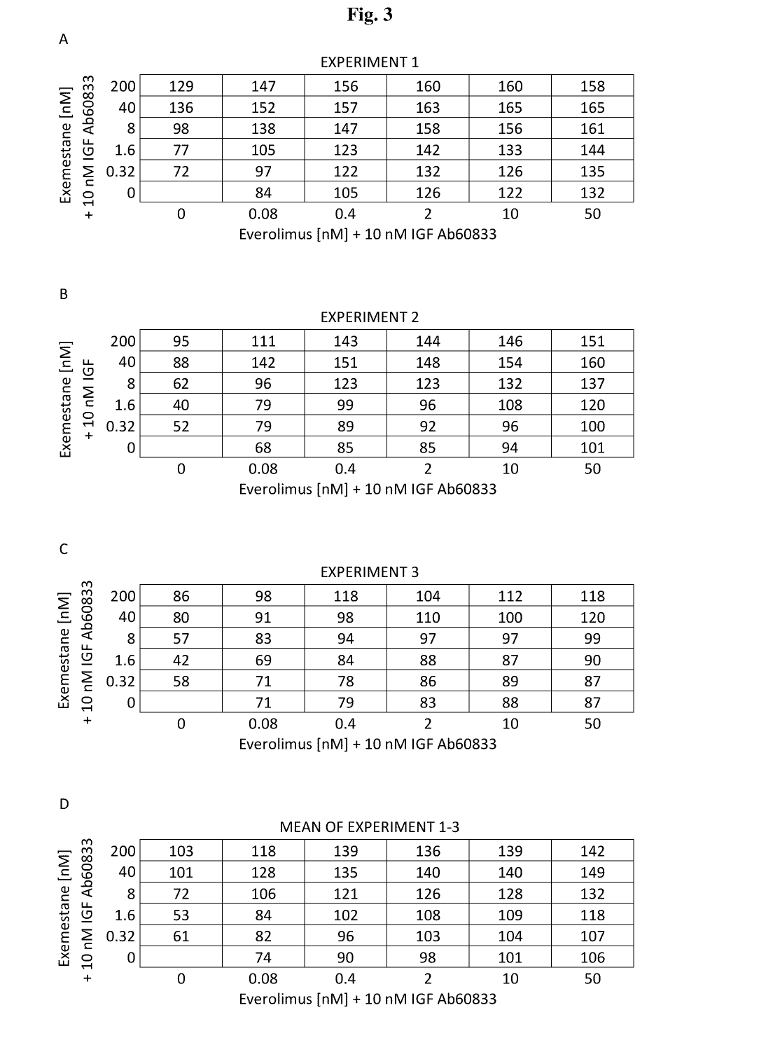

The data from the individual experiments and the mean CGI values are shown in FIG. 3.

### Effect on AKT and S6 Phosphorylation in MCF7aro Cells

The effect of IGF Ab 60833 and everolimus, as single agents and in combination, on the phosphorylation of AKT at serine 473 (pAKT S473) and threonine 308 (pAKT T308), and of S6 (pS6) at serine 235/236 was monitored by Western blot analysis of MCF7aro cell lysates prepared 24 hours post treatment.

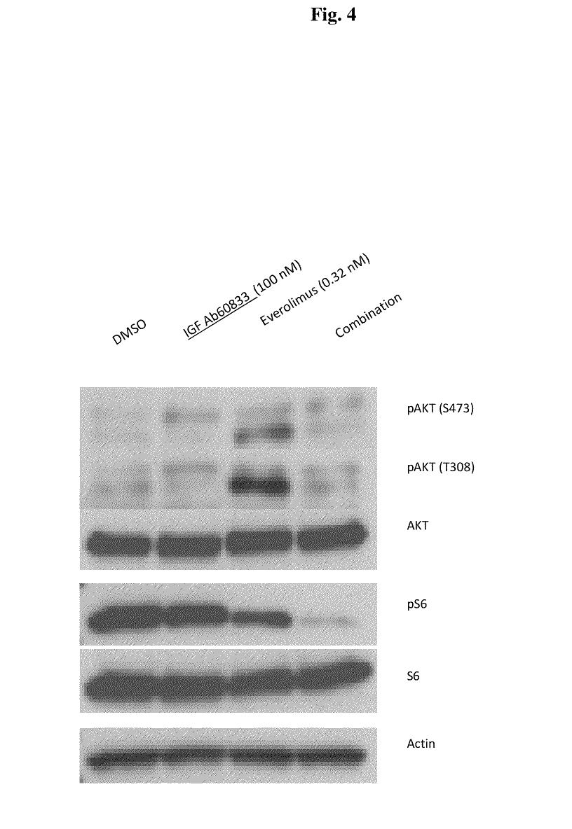

Treatment with IGF Ab 60833 only at 100 nM did not result in inhibition of AKT or S6 phosphorylation. Upon treatment with everolimus at 0.32 nM, a reduced pS6 signal was observed, whereas pAKT S473 and T308 levels increased compared to the vehicle (DMSO)-treated control. Combined treatment with 100 nM IGF Ab 60833 and 0.32 nM everolimus further reduced pS6 levels, i.e. only a weak signal was detectable, and resulted in pAKT S473 and T308 levels comparable to DMSO-treated control (FIG. 4).

### Induction of Apoptosis in MCF7aro Cells

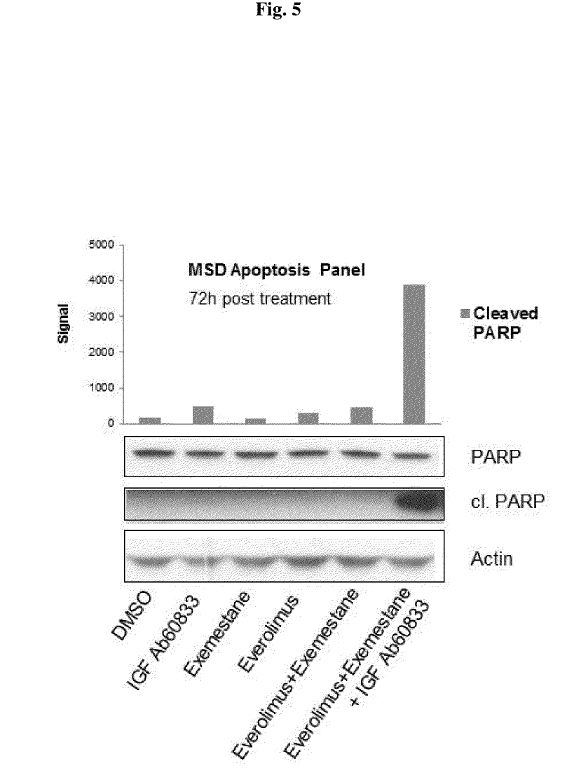

Cleaved PARP as a marker for apoptosis was analysed by Western blotting and MSD Apoptosis Panel in MCF7aro cell lysates prepared after treatment with everolimus, exemestane and IGF Ab 60833, as single agents or in combination, for 72 h. Levels of cleaved PARP after treatment with the single agents or the combination of everolimus and exemestane were comparable to or only marginally higher than the level in the vehicle-treated control. Treatment with the triple combination of everolimus, exemestane and IGF Ab 60833 resulted in a strong induction of cleaved PARP (FIG. 5).

### Discussion

Despite the fact that rapamycin analogues such as everolimus have shown clinical activity in several cancers, pre-clinical and clinical data suggest that there may be acquired resistance by release of a negative feedback loop when mTORC1 is blocked. Following mTORC1 activation a feedback mechanism results in phosphorylation of IRS-1 which is in turn degraded, thereby inhibiting the signalling pathway. Conversely, when mTORC1 is blocked by rapamycin or a rapalog, the negative feedback mechanism is released and the pathway upstream of mTORC1 is re-activated, resulting in increased AKT phosphorylation.

In mice, rapamycin was found to increase the serum IGF bioactivity suggesting that elevated blood IGF levels may at least in part account for the elevated pAKT levels induced by rapamycin. In models of Ewing's sarcoma, IGF Ab 60833 was shown to inhibit the rapamycin-induced increase in pAKT. Together, these findings suggest that the pAKT elevation upon rapamycin treatment was due to enhanced IGF ligand driven signalling. In vivo, the combination of IGF Ab 60833 and rapamycin displayed stronger anti-tumour activity than either single agent alone.

These preclinical studies demonstrated that combination of a rapalog with IGF Ab 60833 leads to a more sustained inhibition of the IGF-1R pathway and can improve efficacy. These data provide a sound rationale for assessing the combination of IGF Ab 60833 with everolimus in estrogen positive breast cancer. Everolimus has recently been approved in combination with the aromatase inhibitor exemestane in this indication.

In the current study, the cellular activity of the triple combination, everolimus, exemestane and IGF Ab 60833, was tested in a human hormone receptor positive breast cancer cell line engineered to express the human aromatase protein (MCF7aro). Under serum-starved conditions, growth of these cells is supported by estrogen which is intracellularly synthesized by the aromatase from androgen supplemented in the growth medium. Using this model, the anti-proliferative activity of aromatase inhibitors can be assessed in vitro.

Treatment of MCF7aro cells with the triple combination resulted in increased cell growth inhibition compared to the dual combination everolimus and exemestane. IGF Ab 60833 reversed everolimus-induced AKT phosphorylation, and the combination led to pronounced inhibition of the IGF-1R signalling pathway. Whereas no cell death was observed after concomitant treatment with everolimus and exemestane, addition of IGF Ab 60833 to the dual combination resulted in induction of apoptosis.

### CONCLUSION

This study has demonstrated that addition of IGF Ab 60833 to the combination of everolimus and exemestane, which is the current standard-of-care treatment in hormone receptor positive breast cancer, leads to improved anti-neoplastic activity in vitro.

1. A method for treating breast cancer which comprises administering to a patient having breast cancer therapeutically effective amounts of an insulin-like growth factor (IGF) receptor antagonist, exemestane and everolimus.

2. The method of claim 1 wherein the breast cancer is locally advanced or metastatic breast cancer.

3. The method of claim 1 wherein the IGF receptor antagonist is an antibody against IGF-1, and antibody against IGF-2, an antibody against both IGF-1 and IGF-2, an antibody against IGF-1 receptor (IGF-1R), or an inhibitor of IGF-1R tyrosine kinase activity.

4. The method of claim 1 wherein the IGF receptor antagonist is an antibody having heavy chain complementary determining regions of SEQ ID NO: 1 (HCDR1), SEQ ID NO: 2 (HCDR2), and SEQ ID NO: 3 (HCDR3) and light chain determining regions of SEQ ID NO: 4 (LCDR1), SEQ ID NO: 5 (LCDR2), and SEQ ID NO: 6 (LCDR3), or an antibody having heavy chain complementary determining regions of SEQ ID NO: 11 (HCDR1), SEQ ID NO: 12 (HCDR2), and SEQ ID NO: 13 (HCDR3) and light chain determining regions of SEQ ID NO: 14 (LCDR1), SEQ ID NO: 15 (LCDR2), and SEQ ID NO: 16 (LCDR3), or an antibody having heavy chain complementary determining regions of SEQ ID NO: 21 (HCDR1), SEQ ID NO: 22 (HCDR2), and SEQ ID NO: 23 (HCDR3) and light chain determining regions of SEQ ID NO: 24 (LCDR1), SEQ ID NO: 25 (LCDR2), and SEQ ID NO: 26 (LCDR3),), or an antibody having heavy chain complementary determining regions of SEQ ID NO: 31 (HCDR1), SEQ ID NO: 32 (HCDR2), and SEQ ID NO: 33 (HCDR3) and light chain determining regions of SEQ ID NO: 34 (LCDR1), SEQ ID NO: 35 (LCDR2), and SEQ ID NO: 36 (LCDR3), or an antibody having a heavy chain variable region of SEQ ID NO: 7 and a light chain variable region of SEQ ID NO: 8, or an antibody having a heavy chain variable region of SEQ ID NO: 17 and a light chain variable region of SEQ ID NO: 18,), or an antibody having a heavy chain variable region of SEQ ID NO: 27 and a light chain variable region of SEQ ID NO: 28,), or an antibody having a heavy chain variable region of SEQ ID NO: 37 and a light chain variable region of SEQ ID NO: 38,), or an antibody having a heavy chain variable region of SEQ ID NO: 41 and a light chain variable region of SEQ ID NO: 42,), or an antibody having a heavy chain variable region of SEQ ID NO: 43 and a light chain variable region of SEQ ID NO: 44, or an antibody having a heavy chain of SEQ ID NO: 9, and a light chain of SEQ ID NO: 10, or an antibody having a heavy chain of SEQ ID NO: 19, and a light chain of SEQ ID NO: 20, or an antibody having a heavy chain of SEQ ID NO: 29, and a light chain of SEQ ID NO: 30, or an antibody having a heavy chain of SEQ ID NO: 39, and a light chain of SEQ ID NO: 40, or an antibody having a heavy chain of SEQ ID NO: 45, and a light chain of SEQ ID NO: 46.

5. The method of claim 4 wherein the antibody has a heavy chain of SEQ ID NO: 39, and a light chain of SEQ ID NO: 40.

6. A pharmaceutical composition, comprising an IGF receptor antagonist, exemestane and everolimus, together with a pharmaceutically acceptable carrier.

7. The method of claim 1 wherein the patient to be treated has one or more characteristics selected from the following: the locally advanced or metastatic breast cancer is positive for estrogen receptor (ER) and/or progesterone receptor (PgR); the locally advanced or metastatic breast cancer is negative for HER2; the locally advanced or metastatic breast cancer is refactory to non-steroidal aromatase inhibitor (e.g. letrozole and/or anastrozole).

8. The method of claim 1 wherein the patient to be treated has the following characteristics: the locally advanced or metastatic breast cancer is positive for estrogen receptor (ER) and/or progesterone receptor (PgR); and the locally advanced or metastatic breast cancer is also negative for HER2; and the locally advanced or metastatic breast cancer is also refactory to non-steroidal aromatase inhibitor (e.g. letrozole and/or anastrozole).
